(19) United States
(12) Patent Application Publication
(10) Pub. No.: US 2024/0074655 A1
(43) Pub. Date: Mar. 7, 2024

(54) SPECIALTY CONTACT LENS DESIGN AND MANUFACTURING

(71) Applicant: Hedgefog Research, Inc., San Pedro, CA (US)

(72) Inventor: Gareguin Mikaelian, San Pedro, CA (US)

(21) Appl. No.: 18/232,773

(22) Filed: Aug. 10, 2023

Related U.S. Application Data
(60) Provisional application No. 63/396,952, filed on Aug. 10, 2022.

(51) Int. Cl. A61B 3/107; A61B 3/10; A61B 3/13; G02C 7/02

(52) U.S. Cl. CPC A61B 3/107; A61B 3/1005; A61B 3/13; G02C 7/027; A61B 3/0058

(57) ABSTRACT

This application discloses systems for the measurement of the shape of the eye and evaluation of a contact lens fit. The measurements of the eye shape can be beneficial for designing specialty contact lenses or for selecting an appropriate contact lens for a specific patient. After the measurement of the shape of the eye is performed, the disclosed systems and methods can be used to analyze the shape, quantify various parameters, and create a simplified graphical representation of the measured eye.

### CROSS-REFERENCE TO RELATED APPLICATIONS

This application claims priority to U.S. Prov. App. No. 63/396,952 filed Aug. 10, 2022 and entitled “OPTOMETRIC INSTRUMENTATION,” which is expressly incorporated by reference herein in its entirety for all purposes.

### BACKGROUND

### Field

The present disclosure generally relates to the field of optometric instrumentation.

### Description of Related Art

The contact lens fitting process is a medical procedure that may include evaluating the patient's vision, ocular surface condition, pathologies, lifestyle, occupation, and other parameters. In general, the eyecare practitioner may consult the patient on the decision of what type of contact lens is suitable. After that the eyecare practitioner may perform a number of evaluations of the ocular surface, optics, and vision and may dispense a trial lens or several trial lenses. Following the initial dispense, the eyecare practitioner may evaluate the lens fit, comfort, and vision, and, if needed, may make appropriate changes and adjustments. In some cases, when, for example, specialty contact lenses are dispensed the eye care practitioner may communicate with a contact lens manufacturer to make changes specifically for a given patient. The contact lens fitting procedure at times requires multiple visits and multiple pieces of optometric examination and diagnostic instrumentation.

In some cases, during a contact lens fitting process, it is advantageous to obtain a three-dimensional representation of the anterior surface of the eye in order to select an appropriate trial lens, or in some cases to manufacture a specialty contact lens, which is specifically designed to fit the patient's eye. There are a number of technologies and commercially available instruments designed to measure the three-dimensional shape of the eye. One of the oldest technologies is based on imaging and analyzing corneal reflections of concentric rings of a Placido disk. This technology typically provides an accurate measurement of the cornea but does not work well with less reflective surfaces of the eye, such as the sclera.

In some cases, the shape of the sclera and cornea is measured using a structured light approach in combination with fluorescent substance applied to the eye. The application of the fluorescent substance allows for the simultaneous imaging of the corneal and scleral surface. In early attempts to use a structured light approach to measuring the three-dimensional profile of the eye, U.S. Pat. No. 4,582,404 to Hamilton discloses an instrument “which measures with great accuracy certain anatomical dimensions of an eye utilizing finite light beams which strike the eye from pre-determined angles and distances.” U.S. Pat. No. 4,995,716 to Warnicki et al., U.S. Pat. No. 5,159,361 to Cambier et al., and U.S. Pat. No. 7,866,820 to Sarver et al. disclose methods of obtaining corneal topography using rasterstereographic images. For example, the '716 patent discloses that “rasterstereographic images of a cornea are produced by staining the cornea with a fluorescein solution which projects a light and dark line pattern onto the cornea through a grid” and “an image processor uses unique software to store and analyze data extracted from the grid pattern.” European Patent No. 0551955 to Jongsma describes a structured light imaging system comprised of two projectors and a single camera. The two projectors project sequences of vertical patterns onto the surface of the eye in such a way that “a flash light source synchronized in sequence with the detection device, and in that the detection device comprises a frame grabber for separately registering a projected grating of each projector in the sequence, respectively, for digital image analysis to obtain the topography of the curved surface.” Later, U.S. Pat. No. 1,115,419 to Kessels et al., discloses several improvements of the Jongsma disclosure including use of Ronchi gratings, slit diaphragm, and telecentric optics. In an alternative approach, U.S. Pat. No. 9,545,200 to Catanzariti et al. discloses a corneo-scleral topographer which uses two cameras and a single projection system.

In other cases, the three-dimensional shape of the eye is measured without the use of fluorescein using scanning or rotating slit, examples of which are disclosed in U.S. Pat. No. 9,489,753, optical coherence tomography, or Scheimpflug imaging technique. Scanning slit technique in combination with Placido topography is implemented in the B+L Orbscan device, examples of which are disclosed in U.S. Pat. No. 6,079,831. The Scheimpflug imaging technique is behind the operating principle of the Pentacam family of products developed and manufactured by OCULUS. These products rely on the Scheimpflug imaging principle, which specifies a geometric relationship between the orientation of the plane of focus, the lens plane, and the image plane.

### SUMMARY

According to a number of implementations, the present disclosure relates to the measurement of the shape, topography, or profilometry of the anterior surface of the eye including the corneal, limbal, and scleral portions. According to a number of implementations, the present disclosure also relates to evaluating the contact lens fit on the eye. According to a number of implementations, the present disclosure also relates to the extraction of parameters of the eye relevant to the contact lens fitting process. According to a number of implementations, the present disclosure also relates to contact lens fitting using these measurements and extracted parameters. According to a number of implementations, the present disclosure also relates to optometric instrumentation used for evaluating various ocular pathologies, evaluating anterior segment shape for contact lens fitting, selecting a contact lens for the eye, evaluating contact lens fit on the eye, and evaluating vision with various types of contact lenses.

According to a first aspect, the present disclosure relates to a system for measuring the three-dimensional map of the anterior surface of the eye.

In some embodiments of the first aspect, the measurement process of the three-dimensional shape of the front surface of the eye can be accomplished with or without application of a fluorescent substance on the surface of the eye.

In some embodiments of the first aspect, the present disclosure provides methods used to shorten the time of the measurement of the eye shape in order to reduce or minimize the errors associated with microsaccadic eye movements and other motion artifacts.

In some embodiments of the first aspect, the present disclosure provides methods for fluorescein-free mapping of the scleral and corneal surface.

In some embodiments of the first aspect, the present disclosure provides a method for combining fluorescent and non-fluorescent slit scanning techniques in order to map the three-dimensional surface of the eye.

In some embodiments of the first aspect, the present disclosure provides a method for combining fluorescent and non-fluorescent structured light imaging in order to map the three-dimensional surface of the eye.

In some embodiments of the first aspect, the present disclosure provides a method for combining fluorescent and non-fluorescent Scheimpflug imaging in order to map the three-dimensional surface of the eye.

According to a second aspect, the present disclosure provides a method for evaluating the fit of a contact lens dispensed on the eye.

In some embodiments of the second aspect, the present disclosure provides a method for evaluating the alignment of the contact lens dispensed on the eye.

In some embodiments of the second aspect, the present disclosure provides a method for evaluating the clearance between the anterior surface of the eye and the back surface of the contact lens dispensed on the eye.

In some embodiments of the second aspect, the present disclosure provides a method for evaluating the alignment of the edge of the contact lens dispensed on the eye with the anterior surface of the eye.

In some embodiments of the second aspect, the present disclosure provides a method for combining fluorescent and non-fluorescent slit scanning techniques in order to evaluate the clearance and alignment between the anterior surface of the eye and the back surface of the contact lens dispensed on the eye.

In some embodiments of the second aspect, the present disclosure provides a method for combining fluorescent and non-fluorescent structured light imaging in order to evaluate the clearance and alignment between the anterior surface of the eye and the back surface of the contact lens dispensed on the eye.

In some embodiments of the second aspect, the present disclosure provides a method for combining fluorescent and non-fluorescent Scheimpflug imaging in order to evaluate the clearance and alignment between the anterior surface of the eye and the back surface of the contact lens dispensed on the eye.

According to a third aspect, the present disclosure provides a method for selecting or designing a contact lens for the eye based on one or more parameters of the eye.

In some embodiments of the third aspect, the present disclosure provides a method for selecting or designing the contact lens to be dispensed on the eye based on parameters measured by the first aspect of the present disclosure or other optometric instruments. The parameters can be acquired using one or more of the following methods: imaging, Placido topography, Scheimpflug imaging, optical coherence tomography, impression molding, structured light scanning, profilometry, slit light scanning, autorefraction measurement, wavefront aberrometry, axial length measurement, or measurement of parameters that characterize visual performance. Additional imaging as well as typical eye and vision measurement techniques can be used for selecting or designing the contact lens to be dispensed on the eye.

In some embodiments of the third aspect, artificial intelligence, such as machine learning algorithms, can be used in the method for selecting or designing the contact lens to be dispensed on the eye.

In some aspects, the techniques described herein relate to a system for measuring a three-dimensional shape of an anterior surface of an eye, the system including: a projector configured to project one or more lines onto a surface of an eye; an imaging system configured to acquire one or more images of the eye with the one or more projected lines, the imaging system being aligned at an alignment angle relative to the projector; and an analysis system configured to analyze the one or more images to determine a shape of the one or more projected lines and to generate a three-dimensional measurement of the eye from one or more images of the projected lines.

In some aspects, the techniques described herein relate to a system, wherein: the projector is configured to project a series of projections in sequence, each projection including a single line projected onto the surface of the eye at a different position from other projections of the series of projections; the imaging system is configured to acquire an image of the single line projected onto the surface of the eye for each projection of the series of projections resulting in a plurality of single line images; and the analysis system is configured to analyze the plurality of single line images to generate the three-dimensional measurement of the eye.

In some aspects, the techniques described herein relate to a system, wherein: the imaging system is configured to acquire a plurality of images; and the projector is configured to project multiple lines onto the surface of the eye during the acquisition of each of the plurality of images.

In some aspects, the techniques described herein relate to a system further including an imaging screen, wherein focusing of the imaging system is achieved by projecting a plurality of lines onto the surface of the eye and aligning the plurality of lines with a plurality of lines displayed on the imaging screen, positions of the plurality of lines being configured such that light from the plurality of lines does not enter the pupil of the eye responsive to the imaging system being in focus.

In some aspects, the techniques described herein relate to a system, wherein the imaging system is configured to image an imaging area that is divided into a plurality of segments, each segment corresponding to a measurement range, and for each image acquired by the imaging system a single line is projected onto each segment.

In some aspects, the techniques described herein relate to a system, wherein the imaging system is configured to image an imaging area that is divided into a plurality of segments, each segment corresponding to a measurement range, and for a portion of the one or more images acquired by the imaging system a single line is projected onto each segment and for another portion of the one or more images acquired by the imaging system multiple lines are projected onto each the segment.

In some aspects, the techniques described herein relate to a system for measuring a three-dimensional shape of a surface of an eye, a corneal thickness of the eye, or clearance between a back surface of a contact lens and the surface of the eye, the system including: a projector having a projector optical axis and configured to project one or more lines onto the surface of the eye; and an imaging system having a lens, an imaging sensor, and an imaging optical axis and configured to image the eye, the imaging system aligned such that the imaging optical axis forms a non-zero angle relative to the projector optical axis, an optical axis of the lens being not normal to the imaging sensor, an angle between the optical axis of the lens and the normal to the imaging sensor being selected so that at least one sheet of light resulting from a single line projected by the projector at a section where the sheet of light intersects structures of the eye is at least partially in focus on the imaging sensor of the imaging system.

In some aspects, the techniques described herein relate to a system, wherein the projector is configured to project lines in a single wavelength band.

In some aspects, the techniques described herein relate to a system, wherein the projector is configured to project lines in more than one wavelength bands.

In some aspects, the techniques described herein relate to a system, wherein a fluorescent substance is applied to the surface of the eye and wherein a long pass optical filter is placed in front or in the optical path of the imaging system.

In some aspects, the techniques described herein relate to a system, wherein a contact lens is placed on the eye; fluorescent substance is applied to one or both the space between the back surface of the contact lens and the front surface of the eye, and wherein a long pass optical filter is placed in front or in the optical path of the imaging system.

In some aspects, the techniques described herein relate to a system, wherein the imaging system is included of multiple imaging sensors, wherein each imaging sensor is configured so that the optical axis of the lens of each the sensor the is not normal to the imaging sensor, and wherein the angle between the optical axis of the lens and the normal to the imaging sensor is selected so that at least one of the sheets of light resulting from a single line projected by the projector at the section where it intersects the structures of the eye is at least partially in focus on the imaging sensor of the second imaging system.

In some aspects, the techniques described herein relate to a system, further including a second imaging system configured such that it is aligned at a certain non-zero angle to the optical axis of the projector and also aligned at a certain non-zero angle with respect to the optical axis of the first imaging system.

In some aspects, the techniques described herein relate to a method for analyzing the three-dimensional shape of the anterior surface of the eye, the method including: obtaining a numerical representation of a three-dimensional shape of the eye; defining a certain subset of the numerical representation; calculating a plurality of parameters representing an approximation of the subset in a series of orthogonal functions; and describing the three-dimensional shape by one or more of the parameters.

In some aspects, the techniques described herein relate to a method, wherein the subset of the numerical representation is a sagittal graph of one or several rings of the surface of the eye.

In some aspects, the techniques described herein relate to a method, wherein the orthogonal functions are Fourier series.

In some aspects, the techniques described herein relate to a method, wherein the subset is the sagittal graph at different angular values and a single cord radius and wherein the orthogonal functions are Fourier series.

In some aspects, the techniques described herein relate to a method, further including: setting a subset of coefficients in the Fourier series to zero; performing an inverse Fourier transform of the remaining Fourier components; and plotting the inverse transform and analyzing the shape of the eye based on the resulting plot.

In some aspects, the techniques described herein relate to a method for selecting a contact lens to be dispensed on the eye, the method including: training a machine learning algorithm using a dataset consisting of plurality of contact lens fits, the dataset containing parameters of successfully and unsuccessfully dispensed lenses and a plurality of parameters and measurements of the eye, the parameters and measurements including but not limited to the three-dimensional shape of the eye; measuring a plurality of parameters of an eye to be fitted with a contact lens; and using the machine learning algorithm to select a lens to be dispensed on the eye.

In some aspects, the techniques described herein relate to a method in claim 19, further including: evaluating the parameters of the contact lens; quantifying the success of the fit; adding the evaluated parameters, the parameters of the eye, and the quantified success of the fit to the dataset; and retraining the machine learning algorithm with a new dataset containing the added data of the lens parameters, the eye parameters, and the fit outcome.

For purposes of summarizing the disclosure, certain aspects, advantages and novel features have been described herein. It is to be understood that not necessarily all such advantages can be achieved in accordance with any particular embodiment. Thus, the disclosed embodiments can be carried out in a manner that achieves or optimizes one advantage or group of advantages as taught herein without necessarily achieving other advantages as can be taught or suggested herein.

### BRIEF DESCRIPTION OF THE DRAWINGS

FIG. 1 illustrates an arrangement of a projector and a camera for linescan or structured light imaging of an eye.

FIG. 2 illustrates various lines projected into the eye and the location of measurement points evaluated during the measurement process.

FIG. 3 illustrates a point cloud of a surface of the eye.

FIG. 4 illustrates an example of an instrument capable of profilometry measurement of the eye surface and Scheimpflug imaging along a horizontal meridian of the eye.

FIG. 5 illustrates a Scheimpflug image of an eye.

FIG. 6 illustrates an instrument capable of Scheimpflug imaging of multiple meridians of the eye.

FIG. 7 illustrates fluorescent Scheimpflug imaging and a combination of fluorescent and non-fluorescent Scheimpflug imaging.

FIG. 8 illustrates two techniques of fluorescent imaging-based evaluation of a contact lens on the surface of the eye with one using vertical lines and one with flat field illumination.

FIG. 9 illustrates a sagittal graph and a graph of the second order or toricity of the sagittal graph.

FIG. 10 illustrates a method of graphical representation of various orders of the Fourier transformation of a sagittal graph.

FIG. 11 illustrates a flowchart of an example method for simplifying the sagittal graph of the eye.

### DETAILED DESCRIPTION OF SOME EMBODIMENTS

The headings provided herein, if any, are for convenience only and do not necessarily affect the scope or meaning of the claims.

### Line Scanning Profilometer

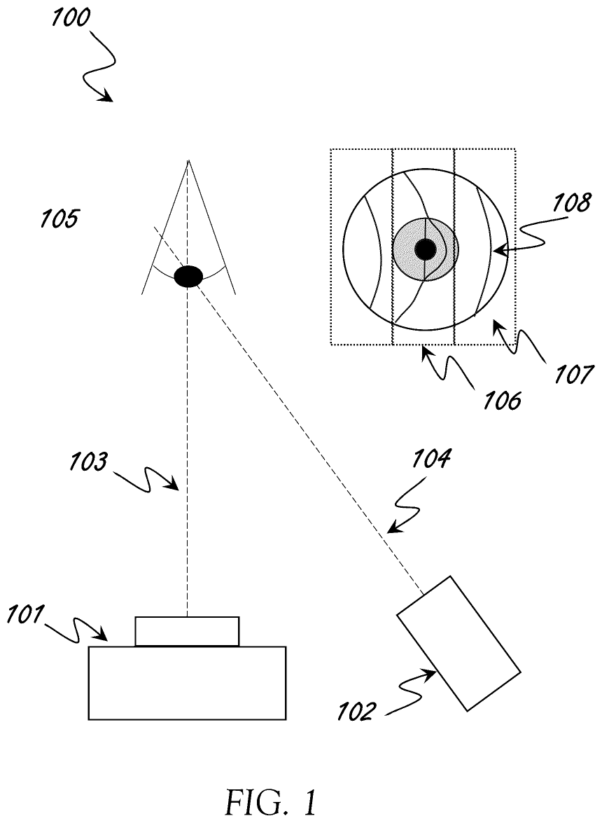

FIG. 1 illustrates a schematic diagram of an example line scanning profilometer 100. The profilometer 100 is configured to determine parameters of an eye 105, such as the three-dimensional shape of the eye 105. The profilometer 100 includes a camera 101 and a projection system 102. The projection system 102 can include a scanning slit, a digital micromirror device (DMD), a digital light processing (DLP) unit, a liquid crystal display (LCD) projector, a laser projector, or any other device capable of projecting lines or patterns onto the surface of the eye. The camera 101 can be any suitable imaging device for capturing image data.

In some embodiments, the camera 101 and the projection system 102 can be arranged so that the optical axis of the camera 101 (the camera optical axis 103) is aligned with the gaze direction of the eye 105 and the optical axis of the projection system 102 (the projector optical axis 104) intersects the camera optical axis 103 at or near a front surface of the eye 105. In this arrangement, lines projected onto the surface of the eye 105 by the projection system 102 can be imaged as curved lines 108 by the camera 101. The curvature of individual curved lines 108 can be analyzed to determine the sagittal height of the eye 105. In particular, the curvature of an individual curved line 108 can be analyzed to determine the sagittal height of the eye 105 at a plurality of points along the curved line 108. To measure the three-dimensional shape of the eye 105, corneo-scleral topography, or eye surface profilometry, the profilometer 100 can scan the projected line (or a plurality of projected lines) across the surface of the eye 105 and can determine a plurality of sagittal height values at a plurality of points. These points can be processed into a point cloud, vector format, g-code, or any other digital format that can be used to represent a three-dimensional shape of an object.

In general, the range of the measurement along the camera optical axis 103 can be limited by the anatomy of the eye 105 being measured. In some embodiments, the range of the measurement along the camera optical axis 103 can be limited and the profilometer 100 can be configured to simultaneously project a plurality of lines onto the surface of the eye 105 to measure the sagittal height at the plurality of points along the plurality of the projected lines. In this approach for measuring the sagittal height, the resulting image 106 of the eye 105 can be divided into a plurality of segments 107 in such a way that a single line projected onto a flat plate positioned at one of the extremes of the measurement range will appear in the image 106 on one side of the segment 107, while the same line projected onto the same plate positioned at the other extreme of the measurement range will appear in the image 106 on the other side of the segment 107. In general, reducing the measurement range of the profilometer 100 may enable an increase in the number of segments 107. A larger number of segments can in turn enable a larger number of projected lines, which in turn can reduce the time required to acquire the map of the ocular surface of the eye 105 with a desired or targeted resolution. In some embodiments, the acquisition time can be reduced by reducing the number of frames projected during a scan by increasing the distance between projected lines in each subsequent frame.

In some implementations, the surface of the eye 105 can be mapped using more than one step. This can be achieved by using the projection system 102 to project a frame with a set of lines such that the projected frame is configured to project a single line onto each segment 107. The projection system 102 can then project one or more frames with a set of lines such that the projected frames are configured to project multiple lines onto each segment 107. To analyze the plurality of images obtained by the camera 101 in this measurement approach, it can be beneficial to make one or several assumptions about the shape of the eye 105. These assumptions may include assumptions about continuity, smoothness, sizes, or any other aspects characterizing the shape of the eye 105. In some cases, the assumptions can be obtained from a normative dataset of eyes or determined based on an analysis of some parameters, pathologies or anatomies of the eye 105 being measured. In certain instances, the first frame projected by the projection system 102 can be used to determine the approximate shape of the eye 105 and the subsequent frames can be used to further analyze the shape of the eye 105 and to improve the accuracy of the measurement. In some cases, the order of the projected images can be different and the projected frame with multiple lines per segment 107 can be projected before the projected frame with a single line per segment 107.

In some implementations, the profilometer 100 can be configured to use the projection system 102 to project a sinusoidal pattern or a plurality of sinusoidal patterns onto the surface of the eye 105 being measured. In such implementations, phase shifting profilometry techniques can be used to determine the shape of the eye 105. In certain implementations, the profilometer 100 can combine phase shifting profilometry with line scanning profilometry. For example, the projection system 102 can be configured to project a plurality of lines to determine the approximate shape of the eye 105 and to project a series of sinusoidal patterns before or after projecting the plurality of lines to improve the accuracy of the determined shape of the eye 105.

In some cases, when the number of points measured on the eye 105 is determined to be less than a desired or targeted resolution, the profilometer 100 can be configured to mathematically or numerically interpolate between measured points to generate a point cloud that has a larger number of points than the number of points acquired during a measurement. This interpolation can include, but not be limited to, cubic splines; Bezier curves; conic sections; higher order polynomials, including but not limited to Zernike, Seidel, or Chebyshev polynomials; Fourier series; spherical harmonics; or any other analytical functions. In some embodiments, one portion of the surface of the eye 105 can be approximated using interpolation, as described herein, while other parts of the surface of the eye 105 can be represented using the measured sagittal heights, as described herein. In certain embodiments, the corneal portion of the surface of the eye 105 can be represented using analytical functions, such as conic sections or Zernike polynomials, while the scleral portion of the surface of the eye 105 can be represented using measured sagittal height values or vice versa.

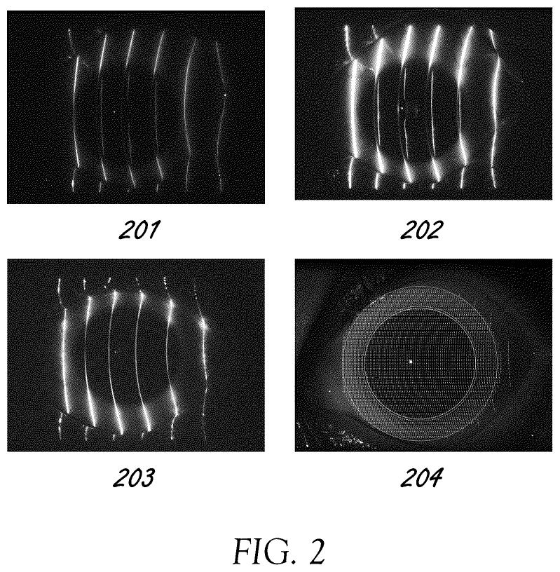

FIG. 2 illustrates examples of images of an eye that can be recorded by a camera of a profilometer, such as the camera 101 of FIG. 1. Image 201 and image 202 are recorded with different settings for the camera and projection system to illustrate different optical properties of different portions of the eye. In image 201, the reflection from the scleral and the iris surfaces are well resolved, but the reflection of the cornea is less visible. There are potential errors that may arise from analysis of image 201, such as missing portions of the corneal map. In image 202 the reflection from the cornea is well resolved, however the scleral image is overexposed and the projected line over the sclera is not well defined. Analyzing this image may result in inaccurate measurement of the sclera.

In order to achieve an accurate scan over different portions of the surface of the eye, it can be beneficial to perform a plurality of scans with different settings for the camera and/or projection system. In some embodiments, two sets of scans can be performed, one set of scans with a high intensity of projected lines and one set of scans with a lower intensity of projected lines. In certain embodiments, two sets of scans can be performed, one set of scans with long exposure of the camera frame and one set of scans with a shorter exposure of the camera frame. In various embodiments, one set of scans can be performed with the lines projected in one wavelength, and another set of scans can be performed with the lines projected in a different wavelength. In the various embodiments, one set of scans can be used to map out one portion of the eye, while another set of scans can be used to map out another portion of the eye. In some implementations, images with brightness characteristics similar to the brightness characteristics of image 201 can be used for mapping the sclera, while images with brightness characteristics similar to the brightness characteristics of image 202 can be used to map the cornea.

In some embodiments, a fluorescent substance can be applied to the eye surface prior to the measurement. One example of a suitable fluorescent substance is fluorescein sodium. When dissolved in the tear film of the eye, this substance absorbs blue light and emits green light. In such embodiments, the lines projected by the projection system 102 can be blue. If a long-pass filter is placed in front of the lens of the camera 101, the blue light reflected by the eye is not imaged and the image recorded by the camera 101 is similar to image 203. For such images, the points over the cornea and sclera can be processed at the same time, simplifying processing and potentially increasing measurement accuracy.

In some implementations, the operator of the instrument can be guided to accurately focus the imaging system (e.g., the camera) on the eye. For example, a line or series of lines can be projected onto the eye surface and a live image overlay can be generated with corresponding lines on the screen of the monitor connected to the imaging device. The operator can then align the images of the projected lines with the overlayed image on the screen. Accurately aligning the projected and overlayed images can result in better focusing. In some embodiments, focusing lines can be projected onto the eye surface so that the focusing lines are configured to not enter the pupil at or near the position of the instrument corresponding to the best focus. This can be done to reduce the amount of light exposure during the focusing process.

Once the measurement is processed, the points which were successfully processed can be illustrated by overlaying the processed points over the image of the eye. An example of such overlayed points is shown in image 204.

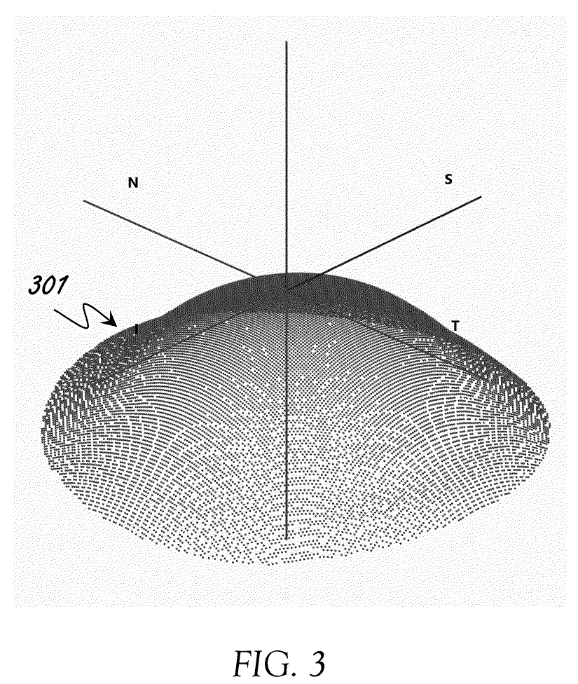

FIG. 3 illustrates another example method of showing a measured point cloud 301 on a screen of a computing device. In some embodiments, the representation of the point cloud 301 can be rotated and subjected to further mathematical analysis. The point cloud 301 can be used for diagnostic purposes or for the purposes of selecting or designing a contact lens for the measured eye.

Line Scanning Profilometer Combined with Scheimpflug Imaging

In some embodiments, it is beneficial to acquire a Scheimpflug image of the eye. The Scheimpflug image can be used to perform corneal pachymetry or corneal thickness measurements. The Scheimpflug image can be combined during processing with profilometry line scans to further improve the measurement accuracy of the disclosed optometric instruments.

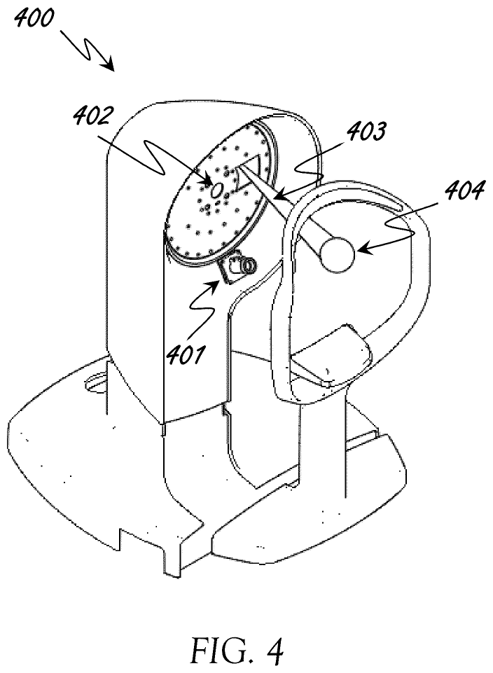

FIG. 4 illustrates an example Scheimpflug imaging device 400. The Scheimpflug imaging device 400 is configured to provide an appropriate or targeted angle between an imaging sensor and a lens plane that can be positioned at a certain distance relative to a camera 402. The Scheimpflug imaging device 400 includes a Scheimpflug imaging unit 401. The Scheimpflug imaging device 400 also includes a projector configured to project a horizontal line or a sheet of light 403 onto an eye surface 404, across the corneal surface, scleral surface, anterior chamber, iris, and crystalline lens.

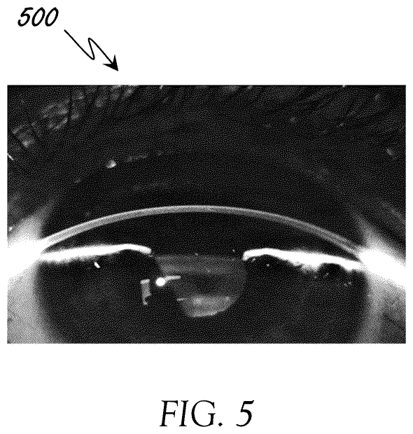

FIG. 5 illustrates an example image 500 that results from imaging a sheet of light with a disclosed Scheimpflug imaging device (e.g., imaging the sheet of light 403 with the Scheimpflug imaging device 400 of FIG. 4). An analysis of the image 500 can reveal information about the corneal thickness, which in some cases can be an indication for corneal pathologies including but not limited to keratoconus and corneal ectasia.

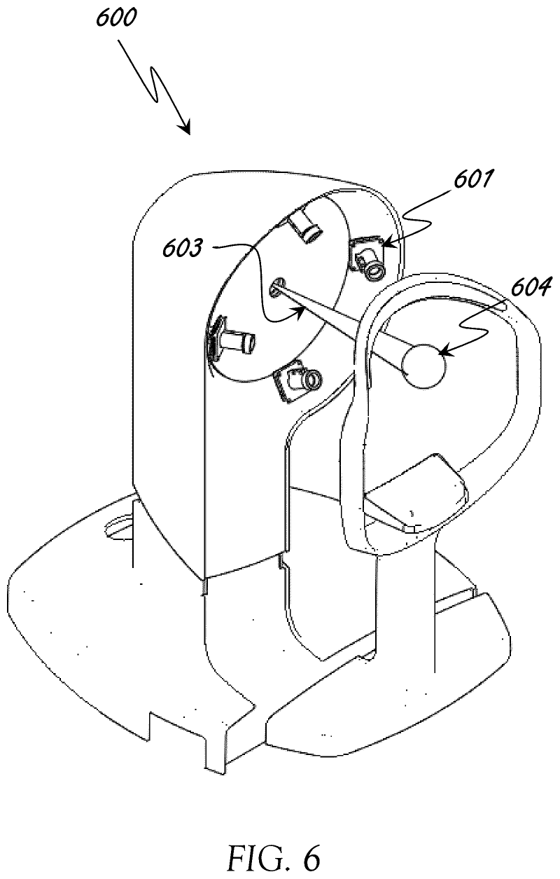

FIG. 6 illustrates another Scheimpflug imaging device 600. The Scheimpflug imaging device 600 includes a projector that is located along an optical axis of the Scheimpflug imaging device 600 and a plurality of Scheimpflug cameras 601 placed in a circular or semi-circular arrangement around the projector. The projector can be programmed to project a series of lines at various angles around the center point of an eye 604, such that each line is projected so that a sheet of light 603 illuminating the eye 604 is parallel to the x-axis of the imaging sensor of at least one of the Scheimpflug cameras 601. During each line projection, a Scheimpflug camera 601 with the imaging sensor's x-axis parallel to the projected sheet of light 603 can be triggered and an image can be recorded by the Scheimpflug camera 601. In some implementations, two Scheimpflug cameras 601 can be used to image the eye 604 in two meridians (e.g., vertical and horizontal), four Scheimpflug cameras 601 can be used to image the eye in four meridians (e.g., 0-180 degrees, 90-270 degrees, 45-225 degrees, 135-315 degrees, etc.), and eight Scheimpflug cameras 601 can be used to image the eye 604 in eight meridians. The number of Scheimpflug cameras can be increased to increase the number of measured meridians. In some embodiments, the Scheimpflug cameras 601 can be positioned on a rotating platform and the number of measured meridians can be increased by rotating the platform by a certain angle or plurality of angles and performing the measurement at each of the plurality of the platform's angles.

Scheimpflug Imaging with and without Fluorescein

In some instances, it can be advantageous to perform Scheimpflug imaging of an eye in fluorescent mode. In the fluorescent mode, a long-pass optical filter can be placed in the imaging path of one or more Scheimpflug cameras. The characteristics of the filter and the projected lines can be chosen so that it will block the wavelength of the projected lines but will transmit longer wavelengths of the light that is emitted by the fluorescent substance applied to the eye. In such instances, it can be beneficial to apply a fluorescent substance to the eye. In certain instances, it can be beneficial to apply the fluorescent substance between the back surface of a contact lens dispensed on the eye and the front surface of the eye. In various instances, it can be beneficial to apply the fluorescent substance to the front surface of the contact lens. In some instances, it can be beneficial to apply the fluorescent substance to both the front and the back surface of the contact lens.

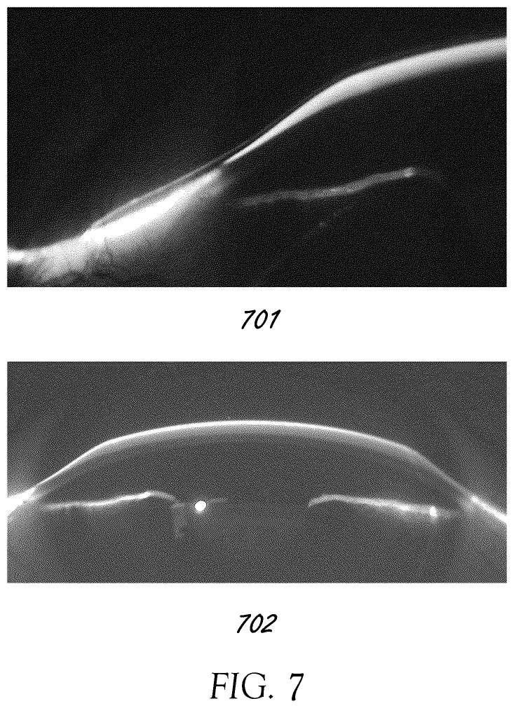

FIG. 7 illustrates an example of a fluorescent Scheimpflug image 701 of a scleral contact lens placed on the eye. In the image 701, a fluorescent substance (e.g., fluorescein sodium) is applied to both the back and the front surface of the contact lens. The eye is illuminated with blue light and imaging is performed through a yellow filter, which transmits green light and blocks blue light.

In some embodiments, it can be beneficial to perform Scheimpflug imaging while illuminating the eye with more than one wavelength. In some implementations, fluorescein sodium can be applied to the eye, a yellow long-pass optical filter can be places in the imaging path of a Scheimpflug camera, and the eye can be illuminated with a line of light simultaneously comprising of green and blue wavelengths. The blue portion of the illuminating light excites fluorescent substance applied to the eye and the green portion of the illuminating light scatters from the eye tissue. By appropriately selecting the relative intensities of the blue and green light it can be possible to simultaneously image the fluorescent substance and the components of the eye. Image 702 is an example of a resulting image acquired in such a fashion.

In some embodiments, it can be beneficial to record two or more Scheimpflug images, with at least one of the images acquired when the eye is illuminated by a line with a wavelength below the passing band of the bandpass filter, and at least one of the images acquired when the eye is illuminated by a line with a wavelength above the passing band of the bandpass filter. Such images can be combined into a single image or separately analyzed.

In some embodiments, the fluorescent portion of the image can be used to evaluate the clearance between the back surface of the contact lens and the eye. This evaluation can be beneficial for properly fitting scleral lenses, ortho-k, lenses, gas permeable corneal lenses, soft contact lenses, or any other types of contact lenses dispensed on the eye.

In some embodiments, it can be beneficial to manufacture a contact lens impregnated or coated with fluorescent substance. When dispensed on the eye and imaged using fluorescent Scheimpflug imaging methods disclosed herein, the fluorescence of the contact lens can be imaged to reveal information about the fit of the contact lens on the surface of the eye. In certain embodiments, the eye can be simultaneously or sequentially illuminated with two wavelengths with longer wavelengths being used to image the structures of the eye and shorter wavelengths being used to image the fluorescent contact lens. By combining the two images, the contact lens and the eye surface can be simultaneously visualized. This type of visualization can be beneficial for evaluating the fit of the contact lens on the surface of the eye.

### Additional Imaging and Measurement Modalities

In some embodiments, the three-dimensional mapping of the eye surface can be combined with other measurements of the eye. In such embodiments, it can be beneficial to design an instrument that can simultaneously or sequentially perform both measurements on the eye.

In some embodiments, the three-dimensional mapping of the eye surface can be combined with video keratography measurements. In such embodiments, it can be beneficial to design an instrument that can simultaneously or sequentially perform both measurements on the eye.

In some embodiments, the three-dimensional mapping of the eye surface can be combined with Placido ring topography measurements. In such embodiments, it can be beneficial to design an instrument that can simultaneously or sequentially perform both measurements on the eye.

In some embodiments, the three-dimensional mapping of the eye surface can be combined with autorefraction measurements. In such embodiments, it can be beneficial to design an instrument that can simultaneously or sequentially perform both measurements on the eye.

In some embodiments, the three-dimensional mapping of the eye surface can be combined with wavefront aberrometry measurements. In such embodiments, it can be beneficial to design an instrument that can simultaneously or sequentially perform both measurements on the eye.

In some embodiments, the three-dimensional mapping of the eye surface can be combined with tear meniscus height measurements. In such embodiments, it can be beneficial to design an instrument that can simultaneously or sequentially perform both measurements on the eye.

In some embodiments, the three-dimensional mapping of the eye surface can be combined with the measurement of the iris diameter and shape. In such embodiments, it can be beneficial to design an instrument that can simultaneously or sequentially perform both measurements on the eye.

In some embodiments, the three-dimensional mapping of the eye surface can be combined with the measurement of the axial length of the eye. In such embodiments, it can be beneficial to design an instrument that can simultaneously or sequentially perform both measurements on the eye.

In some embodiments, the three-dimensional mapping of the eye surface can be combined with the measurement of one or more of the pupil diameter, position, or shape. In such embodiments, it can be beneficial to design an instrument that can simultaneously or sequentially perform both measurements on the eye.

In some embodiments, the three-dimensional mapping of the eye surface can be combined with color imaging. In such embodiments, it can be beneficial to design an instrument that can simultaneously or sequentially perform both measurements on the eye. In some implementations, a monochrome camera can be used to obtain a color image. This can be achieved by sequentially illuminating the eye with several wavelengths, acquiring a plurality of images, and combining the images into a single image or several color images of the eye.

In some embodiments, the three-dimensional mapping of the eye surface can be combined with fluorescence imaging. In such embodiments, it can be beneficial to design an instrument that can simultaneously or sequentially perform both measurements on the eye.

In some embodiments, the three-dimensional mapping of the eye surface can be combined with infrared or visible wavelength imaging of the meibomian glands. In such embodiments, it can be beneficial to design an instrument that can simultaneously or sequentially perform both measurements on the eye.

### Exemplary Methods to Evaluate Lens Fit and Alignment

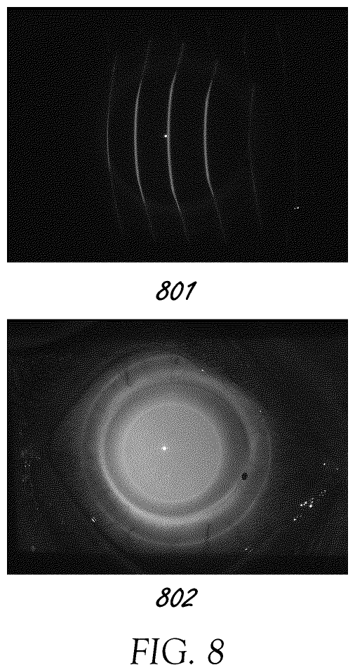

In some embodiments, one or several projected lines can be used to evaluate a contact lens fit on the eye. FIG. 8 illustrates an image 801 of a contact lens disposed on the surface of the eye wherein fluorescent substance is applied between the lens and the eye surface and a plurality of lines is projected onto the eye. The analysis of the width and intensity of such lines may reveal the clearance between the lens and the eye. The shape of the lines may reveal the alignment between the cornea and the back surface of the lens. The locations of the ends of the lines with respect to the eye anatomy may reveal information about the bearing location of the lens on the eye surface.

In some embodiments, the eye can be illuminated with a uniform light of a shorter wavelength and the fluorescent pattern on the eye can be imaged, an example of which is shown as image 802. In some embodiments, the analysis of image 801 and image 802 can be combined. For example, 801 can be used to calculate the relation of the intensity of the fluorescence to clearance and image 802 can be used to generate a clearance map.

In some embodiments, it can be beneficial to combine two or more imaging or measurement techniques to evaluate the fit of the contact lens on the eye. For example, combining anterior segment imaging and Placido topography may reveal information about the contact lens alignment and tilt. As another example, combining fluorescent imaging with color imaging may reveal information about contact lens alignment, impingement, and tear exchange.

### Example Methods for Characterization of an Eye and Contact Lens Selection

The eye care practitioner may have a set of lenses that they can use for selection of a contact lens to be dispensed on an eye. In some cases, the practitioner may select a contact lens from a trial set and prescribe it to a patient without any modifications. In other cases, the practitioner may modify some parameters of the contact lens selected from a trial set, the parameters including, but not limited to, lens diameter, power, cylinder and axis, eccentricity of the optic zone, alignment of the optic zone, higher order aberrations, shape of the back surface, back surface toricity, and others. The practitioner may communicate to the lens manufacturers the initial contact lens selected from the trial set and any specific modifications that are needed for a specific contact lens for a patient being fit with a specialty contact lens.

The eye care practitioner may select the first contact lens to be evaluated on the eye using the manufacturer's recommendation or their own preferences. The eye care practitioner may rely on the measurement of one or a few parameters of the eye to select the initial contact lens to be fitted on the eye. Such parameters may include, but not limited to: the 3-dimensional shape of the eye; corneal k-values; horizontal iris diameter; vertical iris diameter; corneal chamber depth; shape of the limbus; corneal angle at the limbus; scleral angle at the limbus; sagittal depth at a particular chord length; scleral toricity; and others.

An example of a parameter that can be used for contact lens fitting can be characterized as scleral irregularity. It can be beneficial to define scleral irregularity with a plurality of numbers describing the amount of the irregularity at various orders. The numbers can be separated into two groups with some of the numbers grouped into a lower order group, while other numbers are grouped into a higher order group.

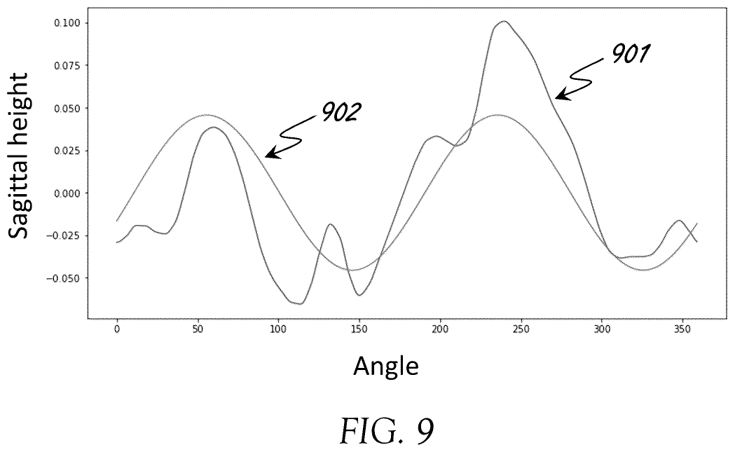

In some embodiments, scleral irregularity can be calculated by selecting one or more chord lengths and plotting a graph of a sagittal height for different angles along the chord length, as illustrated by the graph 901 of FIG. 9. The curve, in general, is a periodic function defined in a range between 0 and 360 degrees. The periodic function can be then analyzed using a Fourier transform, fast Fourier transform (FFT), or other method intended for decomposition of a periodic function into spectral components.

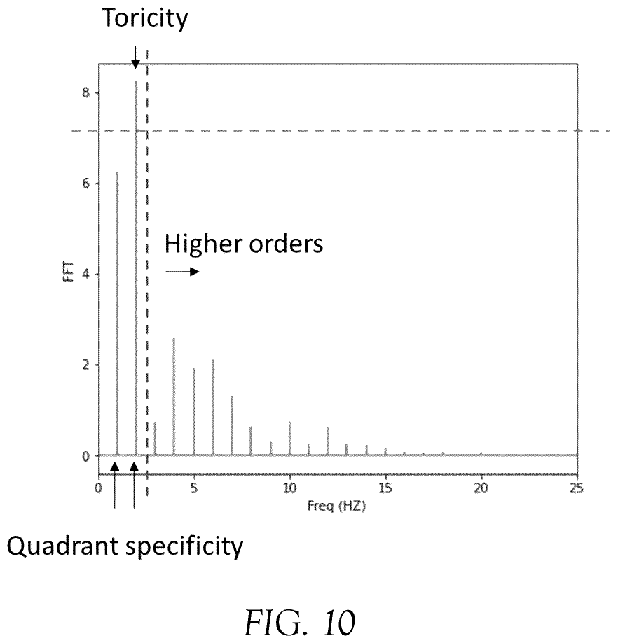

FIG. 10 illustrates decomposition of a sagittal height graph into various spectral components using FFT. It can be beneficial to group the first two components into lower order components, and to group the third order and higher components into higher order components. In some embodiments, the value of the first FFT component can be defined as the tilt and the value of the second FFT component can be defined as the scleral toricity. A mathematical expression can be generated combining the first and the second component and to define the mathematical expression as the quadrant specificity. The mathematical expression can be a sum, quadratic sum, average, weighted average, or any other combination of the first two components of the FFT. Likewise, it can be beneficial to define a mathematical expression that combines the higher order components of the FFT of the sagittal height map. This mathematical expression can be defined as scleral irregularity. The mathematical expression for scleral irregularity can be a sum, quadratic sum, average, weighted average, or any other function combining higher order components of the FFT. The expression can be defined for all higher order components or for a subset of the components.

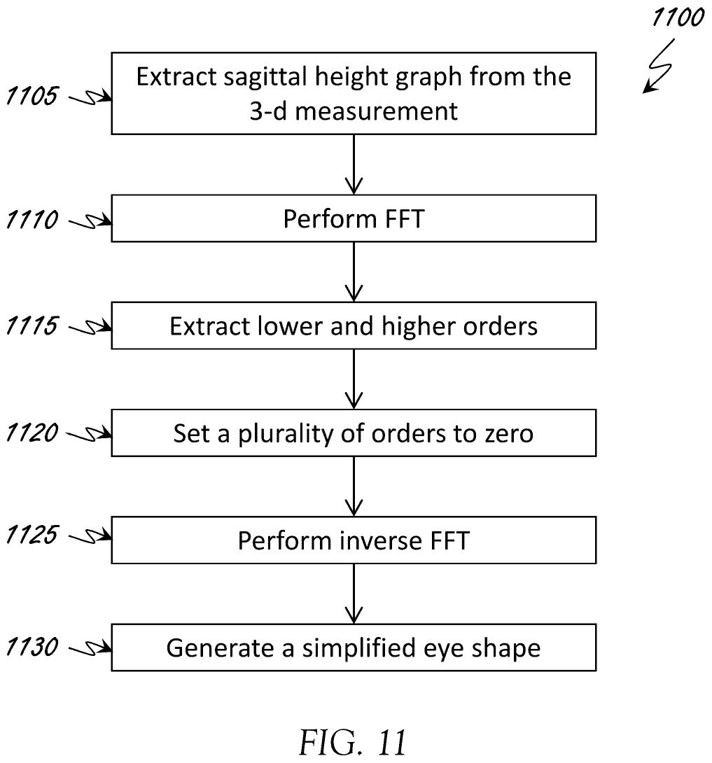

The shape of the eye can be simplified by mathematically removing one or more of the orders of the FFT followed by an inverse Fourier transform or a similar algorithm. An example of such an algorithm 1100 is illustrated in FIG. 11. The algorithm 1100 includes in block 1105 extracting a sagittal height graph from one or more three-dimensional measurements of the eye, examples of which are disclosed herein. In block 1110, the algorithm includes performing a FFT on the sagittal height graph. In block 1115, the algorithm extracts lower and higher orders from the FFT. In block 1120, the algorithm sets a plurality of orders to zero. In block 1125, the algorithm performs an inverse FFT on the non-zero orders. In block 1130, the algorithm generates a simplified eye shape based on the inverse FFT.

As an example of the algorithm 1100, the sagittal graph 901 of the eye can be decomposed into a Fourier series and all but the second components can be set to zero. Then an inverse Fourier transform can be performed where only the toric component of the sagittal height graph remains. If such a transformation is performed on a plurality of chord lengths, a simplified eye model can be generated. In some implementations, if only the second order terms are set to be non-zero, the eye model will be a toric model of the eye, as illustrated in graph 902 of FIG. 9. If the first and second terms are allowed to remain non-zero, a quadrant specific model of an eye is generated.

In some embodiments, it can be beneficial to use one or more measured or calculated parameters of the eye to select a contact lens to be dispensed on the eye. For example, a sagittal height of the eye can be matched to a sagittal depth of the contact lens. In another example, the toricity value at one or several chord lengths can be used to appropriately select the contact lens dispensed on the eye.

In some implementations, multiple parameters of the eye can be used in combination to select a contact lens to be dispensed on the eye. A mathematical algorithm can be developed that takes multiple parameters as an input and the output can include one or more contact lenses that can be dispensed on the eye. In some embodiments, the mathematical algorithm can be an algorithm from the machine learning (ML) family of algorithms. The ML algorithm can be trained on a plurality of lens fits. In some embodiments, the ML algorithm can be trained on a subset of available data. For instance, the ML algorithm can be trained on a dataset specific to a particular eye care practitioner. In certain embodiments, the ML algorithm can be trained on a set of data belonging to a certain demographic. In various embodiments, the ML algorithm can be trained on a subset of data belonging to a patient population sharing a certain pathology. In various embodiments, the ML algorithm can be continuously or intermittently trained as new training data becomes available.

### Additional Embodiments and Terminology

The present disclosure describes various features, no single one of which is solely responsible for the benefits described herein. It will be understood that various features described herein can be combined, modified, or omitted, as would be apparent to one of ordinary skill. Other combinations and sub-combinations than those specifically described herein will be apparent to one of ordinary skill, and are intended to form a part of this disclosure. Various methods are described herein in connection with various flowchart steps and/or phases. It will be understood that in many cases, certain steps and/or phases can be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases can be omitted entirely. Also, the methods described herein are to be understood to be open-ended, such that additional steps and/or phases to those shown and described herein can also be performed.

Some aspects of the systems and methods described herein can advantageously be implemented using, for example, computer software, hardware, firmware, or any combination of computer software, hardware, and firmware. Computer software can comprise computer executable code stored in a computer readable medium (e.g., non-transitory computer readable medium) that, when executed, performs the functions described herein. In some embodiments, computer-executable code is executed by one or more general purpose computer processors. A skilled artisan will appreciate, in light of this disclosure, that any feature or function that can be implemented using software to be executed on a general purpose computer can also be implemented using a different combination of hardware, software, or firmware. For example, such a module can be implemented completely in hardware using a combination of integrated circuits. Alternatively or additionally, such a feature or function can be implemented completely or partially using specialized computers designed to perform the particular functions described herein rather than by general purpose computers.

Multiple distributed computing devices can be substituted for any one computing device described herein. In such distributed embodiments, the functions of the one computing device are distributed (e.g., over a network) such that some functions are performed on each of the distributed computing devices.

Some embodiments can be described with reference to equations, algorithms, and/or flowchart illustrations. These methods can be implemented using computer program instructions executable on one or more computers. These methods may also be implemented as computer program products either separately, or as a component of an apparatus or system. In this regard, each equation, algorithm, block, or step of a flowchart, and combinations thereof, can be implemented by hardware, firmware, and/or software including one or more computer program instructions embodied in computer-readable program code logic. As will be appreciated, any such computer program instructions can be loaded onto one or more computers, including without limitation a general purpose computer or special purpose computer, or other programmable processing apparatus to produce a machine, such that the computer program instructions which execute on the computer(s) or other programmable processing device(s) implement the functions specified in the equations, algorithms, and/or flowcharts. It will also be understood that each equation, algorithm, and/or block in flowchart illustrations, and combinations thereof, can be implemented by special purpose hardware-based computer systems which perform the specified functions or steps, or combinations of special purpose hardware and computer-readable program code logic means.

Furthermore, computer program instructions, such as embodied in computer-readable program code logic, may also be stored in a computer readable memory (e.g., a non-transitory computer readable medium) that can direct one or more computers or other programmable processing devices to function in a particular manner, such that the instructions stored in the computer-readable memory implement the function(s) specified in the block(s) of the flowchart(s). The computer program instructions may also be loaded onto one or more computers or other programmable computing devices to cause a series of operational steps to be performed on the one or more computers or other programmable computing devices to produce a computer-implemented process such that the instructions which execute on the computer or other programmable processing apparatus provide steps for implementing the functions specified in the equation(s), algorithm(s), and/or block(s) of the flowchart(s).

Some or all of the methods and tasks described herein can be performed and fully automated by a computer system. The computer system may, in some cases, include multiple distinct computers or computing devices (e.g., physical servers, workstations, storage arrays, etc.) that communicate and interoperate over a network to perform the described functions. Each such computing device typically includes a processor (or multiple processors) that executes program instructions or modules stored in a memory or other non-transitory computer-readable storage medium or device. The various functions disclosed herein can be embodied in such program instructions, although some or all of the disclosed functions may alternatively be implemented in application-specific circuitry (e.g., ASICs or FPGAs) of the computer system. Where the computer system includes multiple computing devices, these devices may, but need not, be co-located. The results of the disclosed methods and tasks can be persistently stored by transforming physical storage devices, such as solid state memory chips and/or magnetic disks, into a different state.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that can be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure can be readily apparent to those skilled in the art, and the generic principles defined herein can be applied to other implementations without departing from the spirit or scope of this disclosure. The teachings of the invention provided herein can be applied to other methods and systems, and are not limited to the methods and systems described above, and elements and acts of the various embodiments described above can be combined to provide further embodiments. Accordingly, the novel methods and systems described herein can be embodied in a variety of other forms; furthermore, various omissions, substitutions and changes in the form of the methods and systems described herein can be made without departing from the spirit of the disclosure. The accompanying claims and their equivalents are intended to cover such forms or modifications as would fall within the scope and spirit of the disclosure.

1. A system for measuring a three-dimensional shape of an anterior surface of an eye, the system comprising:
a projector configured to project one or more lines onto a surface of an eye;
an imaging system configured to acquire one or more images of the eye with the one or more projected lines, the imaging system being aligned at an alignment angle relative to the projector; and
an analysis system configured to analyze the one or more images to determine a shape of the one or more projected lines and to generate a three-dimensional measurement of the eye from one or more images of the projected lines.

2. The system of claim 1, wherein:
the projector is configured to project a series of projections in sequence, each projection including a single line projected onto the surface of the eye at a different position from other projections of the series of projections;
the imaging system is configured to acquire an image of the single line projected onto the surface of the eye for each projection of the series of projections resulting in a plurality of single line images; and
the analysis system is configured to analyze the plurality of single line images to generate the three-dimensional measurement of the eye.

3. The system of claim 1, wherein:
the imaging system is configured to acquire a plurality of images; and
the projector is configured to project multiple lines onto the surface of the eye during the acquisition of each of the plurality of images.

4. The system of claim 3 further comprising an imaging screen, wherein focusing of the imaging system is achieved by projecting a plurality of lines onto the surface of the eye and aligning the plurality of lines with a plurality of lines displayed on the imaging screen, positions of the plurality of lines being configured such that light from the plurality of lines does not enter the pupil of the eye responsive to the imaging system being in focus.

5. The system of claim 3, wherein the imaging system is configured to image an imaging area that is divided into a plurality of segments, each segment corresponding to a measurement range, and for each image acquired by the imaging system a single line is projected onto each segment.

6. The system of claim 3, wherein the imaging system is configured to image an imaging area that is divided into a plurality of segments, each segment corresponding to a measurement range, and for a portion of the one or more images acquired by the imaging system a single line is projected onto each segment and for another portion of the one or more images acquired by the imaging system multiple lines are projected onto each the segment.

7. A system for measuring a three-dimensional shape of a surface of an eye, a corneal thickness of the eye, or clearance between a back surface of a contact lens and the surface of the eye, the system comprising:
a projector having a projector optical axis and configured to project one or more lines onto the surface of the eye; and
an imaging system having a lens, an imaging sensor, and an imaging optical axis and configured to image the eye, the imaging system aligned such that the imaging optical axis forms a non-zero angle relative to the projector optical axis, an optical axis of the lens being not normal to the imaging sensor, an angle between the optical axis of the lens and the normal to the imaging sensor being selected so that at least one sheet of light resulting from a single line projected by the projector at a section where the sheet of light intersects structures of the eye is at least partially in focus on the imaging sensor of the imaging system.

8. The system of claim 7, wherein the projector is configured to project lines in a single wavelength band.

9. The system of claim 7, wherein the projector is configured to project lines in more than one wavelength bands.

10. The system of claim 7, wherein a fluorescent substance is applied to the surface of the eye and wherein a long pass optical filter is placed in front or in the optical path of the imaging system.

11. The system of claim 7, wherein a contact lens is placed on the eye; fluorescent substance is applied to one or both the space between the back surface of the contact lens and the front surface of the eye, and wherein a long pass optical filter is placed in front or in the optical path of the imaging system.

12. The system of claim 7, wherein the imaging system is comprised of multiple imaging sensors, wherein each imaging sensor is configured so that the optical axis of the lens of each the sensor the is not normal to the imaging sensor, and wherein the angle between the optical axis of the lens and the normal to the imaging sensor is selected so that at least one of the sheets of light resulting from a single line projected by the projector at the section where it intersects the structures of the eye is at least partially in focus on the imaging sensor of the second imaging system.

13. The system of claim 7, further comprising a second imaging system configured such that it is aligned at a certain non-zero angle to the optical axis of the projector and also aligned at a certain non-zero angle with respect to the optical axis of the first imaging system.

14. A method for analyzing the three-dimensional shape of the anterior surface of the eye, the method comprising:
obtaining a numerical representation of a three-dimensional shape of the eye;
defining a certain subset of the numerical representation;
calculating a plurality of parameters representing an approximation of the subset in a series of orthogonal functions; and
describing the three-dimensional shape by one or more of the parameters.

15. The method of claim 14, wherein the subset of the numerical representation is a sagittal graph of one or several rings of the surface of the eye.

16. The method of claim 14, wherein the orthogonal functions are Fourier series.

17. The method of claim 14, wherein the subset is the sagittal graph at different angular values and a single cord radius and wherein the orthogonal functions are Fourier series.

18. The method of claim 17, further comprising:
setting a subset of coefficients in the Fourier series to zero;
performing an inverse Fourier transform of the remaining Fourier components; and
plotting the inverse transform and analyzing the shape of the eye based on the resulting plot.

19. (canceled)

20. (canceled)
